(19) United States
(12) Patent Application Publication
(10) Pub. No.: US 2009/0208569 A1
(43) Pub. Date: Aug. 20, 2009

(54) Homogeneous, Thermoreversible Alginate Films and Soft Capsules Made Therefrom

(75) Inventors: James J. Modliszewski, Brick, NJ (US); Arthur D. Ballard, Westport, ME (US); Christopher J. Sewall, Hope, ME (US); William R. Blakemore, Topsham, ME (US); Peter J. Riley, Yardley, PA (US)

Correspondence Address: PATENT ADMINISTRATOR;FMC CORPORATION, 1735 MARKET STREET, PHILADELPHIA, PA, 19103, US

(73) Assignee: FMC CORPORATION, Philadelphia, PA (US)

(21) Appl. No.: 12/403,993

(22) Filed: Mar. 13, 2009

Related U.S. Application Data
(63) Continuation of application No. 10/824,919, filed on Apr. 14, 2004, now abandoned.
(60) Provisional application No. 60/462,617, filed on Apr. 14, 2003.
(60) Provisional application No. 60/462,758, filed on Apr. 14, 2003.
(60) Provisional application No. 60/462,721, filed on Apr. 14, 2003.
(60) Provisional application No. 60/462,785, filed on Apr. 14, 2003.
(60) Provisional application No. 60/462,783, filed on Apr. 14, 2003.
(60) Provisional application No. 60/462,794, filed on Apr. 14, 2003.
(60) Provisional application No. 60/462,792, filed on Apr. 14, 2003.
(60) Provisional application No. 60/462,793, filed on Apr. 14, 2003.

(51) Int. Cl. A61K 9/64; A23L 1/00; B32B 5/16; B01J 13/02; C09D 103/00; C09D 101/00; C09D 101/26; C09D 17/00

(52) U.S. Cl. 424/456; 426/89; 102/502; 264/4.1; 106/162.1; 106/163.01; 106/206.1; 106/172.1; 106/501.1; 106/194.2; 106/215.5; 106/203.2; 106/205.72; 106/205.01; 106/217.7

(57) ABSTRACT

The present invention is directed to a homogeneous, thermoreversible gel film comprising a film forming amount of a water soluble, thermoreversible alginate, and optionally at least one of a plasticizer, a second film former, a bulking agent, and a pH controlling agent; and processes for the preparation thereof. The present invention is also directed to soft capsules and solid forms containing the gel film, as well as processes for the preparation thereof.

### RELATED APPLICATIONS

This application claims the benefit of U.S. Provisional Application No. 60/462,617, filed on Apr. 14, 2003.

### FIELD OF THE INVENTION

The present invention is directed to a homogeneous, thermoreversible gel film comprising a film forming amount of a water soluble, thermoreversible alginate, and optionally at least one of a plasticizer, a second film former, a bulking agent, and a pH controlling agent; and processes for the preparation thereof. The present invention is also directed to soft capsules and solid forms containing the gel film, as well as processes for the preparation thereof.

### BACKGROUND OF THE INVENTION

Gelatin has long been used to form films useful in the preparation of films and soft capsules. It is a hydrolyzed protein from collagen usually obtained by boiling animal bones and cartilage under pressure with water. However, the use of gelatin suffers from several commercial drawbacks; e.g., its animal origins often preclude its availability to those who cannot or will not take animal derived capsules and recent concerns over bovine spongiform encephalopathy, BSE, or “Mad Cow Disease.”

As a result, academia and industry have been trying many years to develop alternatives to gelatin that can desirably use many of the machines and processes, such as rotary dies, that are already in place to make soft capsules from gelatin alternatives.

For example, Japanese Patent Application Kokai Publication No. 61-10508A discloses capsules made from the composition of polysaccharides including at least carrageenan and polyhydric alcohols. Carrageenan can be used wholly or partly with other polysaccharides such as tamarind gum, pectin, gelatin, alginates, agar, furcellaran, cellulose derivatives, locust bean gum, and guar gum. Polyhydric alcohols include sorbitol, glucose, sucrose, ethylene glycol, diethylene glycol, triethylene glycol, polyethylene glycol, propylene glycol, polypropylene glycol, butane diol and glycerin. The soft capsules are made from concave stamping dies.

Japanese Patent Application Kokai Publication No. 63-164858 discloses mixtures of polysaccharides and polyhydric alcohols with/without alkaline substances. The broad list of polysaccharides purported to be useful in the application include natural polysaccharides such as carrageenan, alginic acid, alginate derivatives, agar, locust bean gum, guar gum, tamarind seed polysaccharides, pectin, xanthan gum, glucomannan, chitin, pullulan and cyclodextrine. The polysaccharides are stated to be combined with a concentrated water solution of at least one of a polyhydric alcohol, sugar alcohol, monosaccharide, disaccharide and oligosaccharide. The mixtures are stated to be useful in forming hulls of soft capsules. The three examples are directed to making hulls of soft capsules having double layers of the mixture with gelatin and a single layer consisting of the mixture of the invention with gelatin. No specific alginates are mentioned.

U.S. Pat. No. 5,089,307 discloses heat-sealable edible films comprising at least a film layer containing a water-soluble polysaccharide as the principal component, a polyhydric alcohol and water. The films are stated to be useful for sealing and packaging materials for dried foods, oily foods and the like. The polysaccharides purported to be useful include alginic acid and its salts (such as sodium salt); furcellaran; carrageenan such as kappa-, iota- and lambda-carrageenans; agar; pectin such as high-methoxy and low-methoxy pectins; gums such as tamarind seed gum, xanthan gum, guar gum, tara seed gum, locust bean gum; pullulan; chitin derivatives such as chitosan; starch such as wheat, corn and potato starches; dextrin; edible water-soluble cellulose derivatives such as carboxymethylcellulose; and mixtures of the foregoing. The weight ratio of the polyhydric alcohol to polysaccharide is preferably used in an amount of 1:5 to 1:1, and the polysaccharide is present in an amount of not less than 50% of the total amount of active components.

U.S. Pat. No. 6,331,205 discloses aqueous viscous compositions for making soft or hard capsules containing carrageenan, preferably, iota carrageenan as the single gelling agent. Iota-, lambda-, mu-, and nu-carrageenans are disclosed. Plasticizers are disclosed such as those belonging to the polyoxyls class; e.g., glycerol, sorbitol, maltodextrins, dextrose, mannitol, xylitol, polyoxyethylene glycol 400 to 6000, natural glycerides and hemisynthetics and their derivatives, etc. Soft capsules are said to be obtained by an adaptation of the “Scherer” method. There is no description of alginates.

U.S. Pat. No. 6,214,376 discloses gelatin-free capsules made from compositions comprising water soluble hydrophilic colloidal layers comprising gel films of kappa-carrageenan and a plasticizer. The gelatin free soft capsules are said to be made from kappa-carrageenan as the main gel-forming polymer (at least 50% by weight of gums that form thermoreversible gels or contribute to the formation of thermoreversible gels). Hydrolyzed starches such as maltodextrin may be added to increase solids concentration, aid heat sealing and prevent hazing induced by gelling salts. Alginates are not disclosed.

U.S. Pat. No. 6,340,473 discloses the use of a modified starch having a hydration temperature below about 90° C. and iota carrageenan for the manufacture of soft capsules using rotary die encapsulation apparatus. The weight ratio of the modified starch to the iota carrageenan is stated to be crucial to forming a satisfactory film. That is, the weight ratio of the modified starch to the iota carrageenan is said to be 1.5:1. The inventors purportedly found that iota-carrageenan alone does not produce an acceptable film and that modified starch alone does not produce an acceptable film useable for encapsulation. The stated theory is that the iota carrageenan functions as an elasticizing agent rendering an otherwise inelastic, modified starch film, elastic.

It is known that certain high solids, low moisture film forming compositions containing, for example, hydrocolloids, form highly viscous solutions that make formation of hydrated films difficult to obtain. The present invention provides a process for preparing high solids, low moisture films from such highly viscous solutions.

In addition, many attempts have been made to make soft capsules from high solids, low moisture films such as hydrocolloids. However, such attempts to make soft capsules have suffered from the drawback mentioned above. That is, hydrocolloids are known to form highly viscous solutions that are difficult to sufficiently hydrate and form a film in conventional soft capsule making processes. The process of the invention therefore allows for the manufacture of soft capsules from such films.

### SUMMARY OF THE INVENTION

As a first embodiment, the present invention is directed to a homogeneous, thermoreversible gel film comprising a film forming amount of a water soluble, thermoreversible alginate and optionally at least one of a plasticizer, a second film former, bulking agent, and a pH controlling agent.

As a second embodiment, the present invention is directed to a process for making the gel films of the invention comprising the steps of: (i) heating, hydrating, mixing, solubilizing, and, optionally, de-aerating a composition of the alginate and optionally at least one of a plasticizer, a second film former, a bulking agent and a pH controlling agent in an apparatus providing sufficient shear, temperature and residence time to form a homogeneous, molten composition, wherein the temperature is at or above the solubilizing temperature of the molten composition; and (ii) cooling the molten composition to or below the gelling temperature of the molten composition to form the gel film.

As a third embodiment, the present invention is directed to soft capsules comprising capsule walls and an encapsulated substance wherein the capsule walls comprise the gel films of the present invention, as a well as a process for the preparation thereof. The process comprises the steps of: (i) heating, hydrating, mixing, solubilizing, and, optionally, de-aerating a composition of the alginate and optionally at least one of a plasticizer, a second film former, a bulking agent and a pH controlling agent in an apparatus capable of providing sufficient shear, temperature and residence time to form a homogeneous, molten composition, wherein the temperature is at or above the solubilizing temperature of the molten mass; and (ii) making soft capsules directly from the molten composition or allowing the molten composition to cool to its gelling temperature or below and thereafter making soft capsules therefrom.

As a fourth embodiment, the present invention is directed to solid forms comprising a fill material encapsulated by the homogeneous, thermoreversible gel film of the present invention; e.g., hard capsules.

### BRIEF DESCRIPTION OF THE DRAWINGS

FIG. 1 is a schematic of a process of the present invention using a Stephan processor together with an extruder.

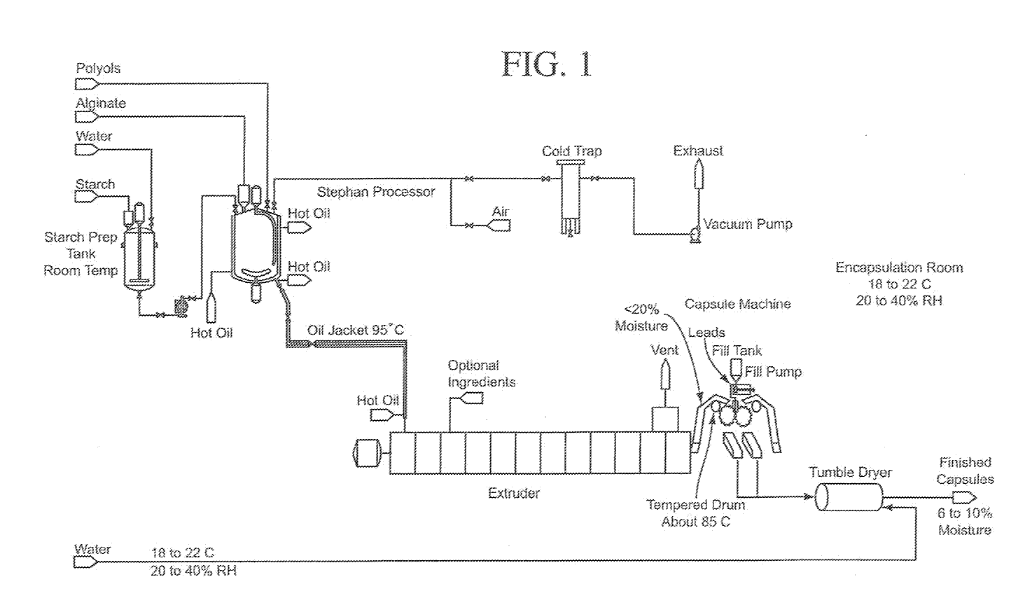

FIG. 2 is a schematic of a process of the present invention using a fluid mixing apparatus of FIG. 3 and an extruder. The schematic shows the film coming out of the extruder proceeding to the encapsulation apparatus.

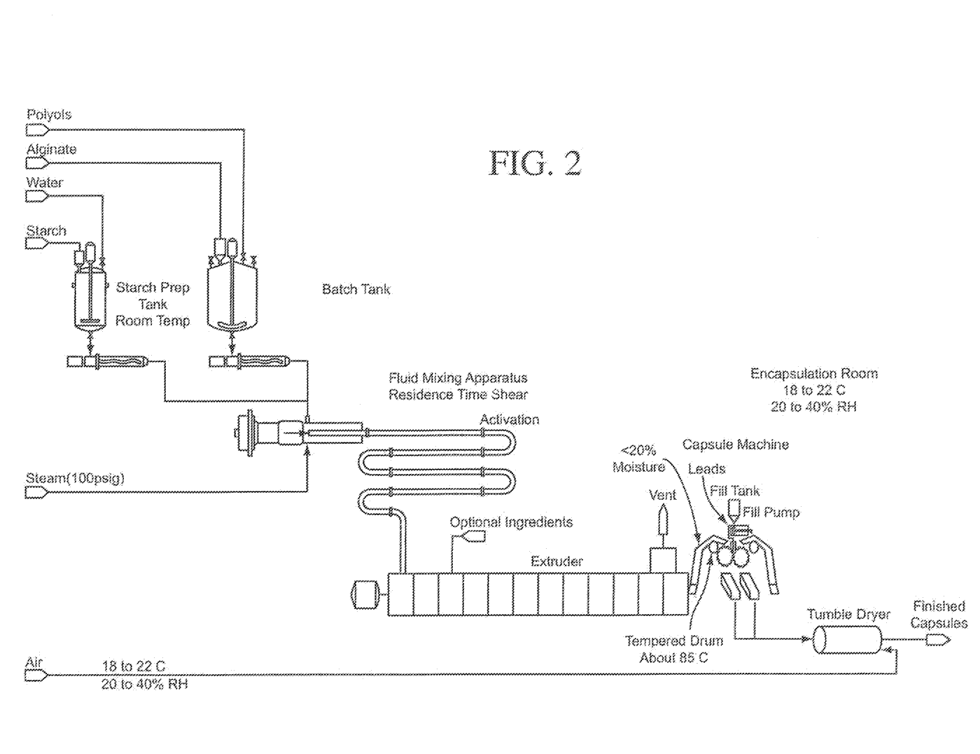

FIG. 3 is a partially broken away, side elevational view of the fluid mixing apparatus for mixing first and second fluids with steam that can be used in the process of the present invention.

FIG. 4 is another version of the schematic of FIG. 2 showing the film coming out of the extruder proceeding to the encapsulation apparatus.

FIG. 5 is a schematic of a process of the present invention using the fluid mixing apparatus of FIG. 3, a cooling drum and an encapsulation apparatus.

### DETAILED DESCRIPTION OF THE INVENTION

Alginates, derived from, inter alia, brown seaweeds (Phaeophyceae sp.) are linear unbranched chemical polymers containing (1-4)-linked β-D-mannuronic acid (M) and α-L-guluronic acid (G) residues. Alginates are not random copolymers, but consist of blocks of similar and alternating residues, for example, MMMM; GGGG, and GMGM, and are generally useful in the form of alginic acid or salts thereof.

Alginates that are useful in the present invention in forming homogeneous, thermoreversible gel films include the monovalent salts of alginate such as sodium and potassium alginate, as well as the esterified forms of alginate such as propylene glycol alginate. All such esterified forms are encompassed within the definition of alginates as used herein. Additional examples of alginates useable in the present invention include magnesium alginate and 3-ethanol amine alginate. All such alginates, as described above and herein, may be used solely or in combination with other alginates of the present invention, and such other alginates may include small amounts of other cations provided such do not, as discussed in more detail below, adversely impact the gel formation or homogeneity of the gel film.

Certain alginates, e.g., sodium alginate, are generally considered to produce thermally irreversible gels with calcium ions. To the inventors' surprise, such alginates, in accordance with the present invention, have been found to produce homogeneous, thermoreversible gel films having significant film strength. In addition, certain alginates, such as propylene glycol alginate, are generally considered to be non-gelling. Also to the inventors' surprise, propylene glycol alginate has been shown to provide a homogeneous, thermoreversible gel film having significant film strength. The alginates may be fully, partially or not clarified from the raw material.

The alginate is used in the present invention in a film forming amount (e.g., an amount that adds strength to the gel film) which is distinguished from trace amounts of alginate that do not add film properties to the film. Thus, for example, in a gel film of the present invention, a film forming amount of alginate is an amount that adds film strength to the overall film. Such film forming amounts are generally at least 0.25% by weight of the dry gel film, particularly, 0.5% to 90%, more particularly, 0.5% to 50%, more particularly, 0.25 to 25% by weight of the dry gel film depending on the application.

Alginate is known to react and crosslink with available polyvalent cations (ionic) such as calcium. This crosslinking can adversely impact the film formation and thermoreversibility of the alginate gel films depending on the polyvalent cation used; e.g., magnesium does not adversely impact the thermoreversibility of the alginate gel film. Therefore, it is important that the alginate have levels of those polyvalent cations that adversely impact the film formation and thermoreversibility of the alginate film below the point that such impair film formation or thermoreversibility within the alginate system. Such polyvalent cation (e.g., calcium) levels are generally used in an amount of no higher than 5.0%, more preferably, less than 2.0%, more preferably, less than 1.0% by weight based on the dry weight of the alginate in the gel film, but this can vary depending on the effect of other components, alginate type and source, and the use of sequestering agents, etc. Sequestering or chelating agents could be added in sufficient amounts to minimize the above adverse polyvalent cation solubility (and participating activity) of e.g., calcium.

The gel films of the present invention are homogeneous, thermoreversible films. As used herein, “homogeneous film” defines films that, to the naked eye, are visually uniform and free of defects such as lumps, cracks, particles that are undissolved that should be dissolved, non-uniform distribution of insoluble particles, etc. “Fish eyes” (mixed liquid and solid states) or “gel balls” (non-uniform gel structure) would not meet the definition of “homogeneous” as used herein.

The gel films of the present invention are homogeneous, thermoreversible gel films. They can be cast and used in a wide variety of applications as cast films or in subsequent processing.

As used herein, “thermoreversible film” defines a film that has a melting temperature. As used herein, the melting temperature is the temperature or temperature range over which the film softens or flows.

As used herein, “gel films” refer to thin membranes formed of structured alginate. The gel forming composition is characterized by a gel temperature, the temperature below which the molten mass must be cooled to form a self supporting structure. Optionally, a molten mass can be cast hot and allowed to cool, as well as dry to further concentrate the solids (controlled moisture removal) until a gel film is formed by the gel composition. The melt temperature of a thermoreversible gel film is higher than its gel temperature.

The homogeneous, thermoreversible gel film suitable for making capsules of the present invention can optionally contain at least one of a plasticizer, a second film former, a bulking agent and a pH controlling agent. The components to be added to the gel film and their amounts can vary depending on the desired use of the alginate gel film.

Examples of such a plasticizer include polyols such as glycerin, sorbitol, maltitol, lactitol, corn starch, fructose, polydextrose, solubilized oil, and polyalkylene glycols such as polyethylene glycol and propylene glycol. The amount of the plasticizer can vary depending on the use of the gel film and its desired elasticity. For example, such plasticizers should generally be used in an amount of at least 5%, more preferably, at least 10%, more preferably, at least 20%, more preferably, at least 30% by weight of all the components including water in the dry film if a gel film having more elasticity is desired; e.g., films to be used to make soft capsules. For other applications, such as hard capsules, where less elastic films are desired, the plasticizer can be present in an amount of 0% to 20% by weight of all the components in the dry film. It is possible that the gel film of the invention contains no plasticizer at all.

Examples of the second film former that can be used in the present invention include at least one of a starch, starch hydrozylate, starch derivative, cellulose gum, hydrocolloid, an alkylcellulose ether or a modified alkyl cellulose ether. Examples of the hydrocolloid include at least one of kappa carrageenan, kappa-2 carrageenan, iota carrageenan, polymannan gums such as locust bean gum or guar gums including low viscosity guar gum, pullulan, gellan (including high and low-acyl gellan), dextran, pectin and less than fully modified versions thereof and combinations thereof. As used herein, kappa-2 carrageenan has a molar ratio of 3:6-anhydrogalactose-2-sulfate (3:6 AG-2S) to 3:6-anhydrogalactose (3:6 AG) content of 25 to 50%, iota carrageenan has a molar ratio of 3:6 AG-25 to 3:6 AG content of 80 to 100% and kappa carrageenan has a molar ratio of 3:6 AG-25 to 3:6 AG content less than that for kappa-2 carrageenan. For example, kappa carrageenan from Eucheuma cottonii, a commonly known and used seaweed source for kappa carrageenan, has a molar ratio of 3:6 AG2S to 3:6 AG content of less than about 10%; and iota carrageenan from Eucheuma Spinosum, a commonly known and used seaweed source for iota carrageenan, has a molar ratio of 3:6 AG2S to 3:6 AG content greater than about 85%. Kappa, kappa-2, and iota carrageenans are different from each other in both structure and functionality. If desired, where iota, kappa or kappa-2 carrageenan is used as the second film former, such carrageenan can have a viscosity of 19 cps or less, more particularly less than 10 cps, at 75° C. in a 0.10 molar sodium chloride solution containing 1.5% of the reduced molecular weight carrageenan based on the total weight of the solution. This viscosity test can be performed using a Brookfield LVF (Brookfield Engineering Laboratories, Inc.) viscometer using Spindle #1 at 60 r.p.m. and determining the viscosity after six revolutions. An example of an alkylcellulose ether that can be used in the present invention is hydroxyethylcellulose. Examples of modified alkylcellulose ethers that can be used in the present invention include hydroxypropylcellulose and hydroxypropylmethyl-cellulose. Note that some second film formers, such as carrageenan, can contain cations that can have positive and negative impacts on gelling properties and film strength with the carrageenan and/or the alginate. Such beneficial cations include potassium, magnesium and ammonium. These cations can be present in the carrageenan or added to it from other organic or inorganic sources. These beneficial cations can be present in an amount of less than 20% by dry weight of the alginate in the gel film (including water). This amount can be varied depending on the components in the system and desired melt and sealing temperatures.

Other cations, such as calcium (as mentioned above), aluminum and chromium can adversely crosslink with alginate and/or impact carrageenan stability and should be kept to a minimum, such as less than 5%, less than 2%, less than 1% by dry weight of the alginate in the dry gel film (including water). The alginate can be the only film former in the gel film. When the gel films of the present invention contain second film formers, the alginate can be present in an amount of at least 10%, at least 40%, at least 60% or at least 80% by weight of the total amount of film formers in the dry gel film.

A dried film is the residual form of a cast film after controlled water removal. Combinations of ingredients, such as: alginate, and, optionally, a starch, a polyol and water for processing, are dispersed, hydrated, solubilized and, optionally, de-aerated within the process options described within. The resulting homogeneous mass is cast or formed at the desired solids level (necessary to achieve the intended end-product). The cast system is formed, via gravitational or controlled forces, and subsequently either immediately further processed (such as soft gel capsule production) or the cast mass is additionally processed by utilizing various methods for uniform and controlled water removal until the desired moisture level is reached. Controlled water removal from the cast system allows a further strengthening/alignment of the homogeneous film ingredients into a denser structure, which can further strengthen film characteristics. Moisture removal is limited to that moisture not bound to the molecular surface of the alginate and any other components. The dried film is achieved when the originally cast film does not lose additional weight while subject to the various drying methods employed in the dewatering/dehydration process. A reduction in moisture content to constant levels also imparts stability to the film and, optionally, its contents (if embedded or enrobed or entrapped, etc.) as water activity is also reduced by the process.

Examples of the bulking agent include microcrystalline cellulose, microcrystalline starch, modified and unmodified starch, starch derivatives, inulin, starch hydrozylates, sugar, corn syrup and polydextrose. As used herein and in the claims, the term “modified starches” includes such starches as hydroxypropylated starches, acid thinned starches and the like. Examples of modified starches that can be used in the present invention include Pure Cote™ B760, B790, B793, B795, M250 and M180, Pure-Dent™ B890 and Pure-Set™ B965, all available from Grain Processing Corporation of Muscatine, Iowa, and C AraTex™ 75701, available from Cerestar, Inc. Examples of starch hydrozylates include maltodextrin also known as dextrin. Unmodified starches such as potato starch can also contribute to the film strength when combined with the hydrocolloids within the scope of the invention. In general, modified starches are products prepared by the treatment of starches, for example, acid treatment starches, enzyme treated starches, oxidized starches, cross-bonding starches and other starch derivatives. It is preferred that the modified starches be derivatized wherein side chains are modified with hydrophilic or hydrophobic groups to thereby form a more complicated structure with a strong interaction between side chains.

The amount of the bulking agent to be used in the present invention is generally in the amount of 0 to 20% by weight of the dry film, but more can be used, if desired, for example, at least 20%, more preferably, at least 30% of the dry film.

Note that starch, starch derivatives and starch hydrozylates can be multifunctional. That is, in addition to being used as bulking agents, they can be used as second film formers. When such are used as bulking agents and second film formers, they are generally used in an amount of at least 10%, preferably, at least 20%, more preferably, at least 30% by weight of the dry gel film depending on the application; e.g., soft capsules.

Examples of the pH controlling agent to be used in the present invention include bases such as hydroxides, carbonates, citrates and phosphates. The pH controlling agent can be used to improve the stability of the gel film. For some compositions containing second film formers, the pH controlling agent can be chosen as the source of added beneficial cations such as potassium or ammonium. The amount of the pH controlling agent is generally in the amount of 0 to 4%, more preferably, 0 to 2% by weight of the dry film.

The dry films (e.g., 80% solids or higher) made from the gels of the present invention have been found to have, for example, a break force strength of at least 2,500 grams, at least 4,000 grams, at least 5,000 grams, and at least 6,000 grams, as determined using a Texture Analyzer TA 108S Mini Film Test Rig. Furthermore, dry films of the invention have been shown to have a break force strength in excess of 7,000 and 8,000 grams.

The gel films of the present invention have been found to have a solids content of at least 50%, at least 60%, at least 70%, at least 80% and at least 90% of all components in the gel film. It is understood that up to 15%, 10%, 5% water may remain strongly associated with the solids in the gel film when dry.

The gel films of the invention can also contain colorants and flavorants such as sugar, corn syrup, fructose, sucrose, etc, whether or not other components, such as plasticizers, bulking agents, second film formers, etc. are present. One embodiment of a gel film of the invention comprises the alginate described herein, flavorant and water in a high solids system; e.g., greater than 50%, 60%, 65%, 75%, 80%, 85%, 90% solids.

Dry film thicknesses generally used for soft capsules are in the range of 0.5 to 3.0 mm, more preferably, 0.8 to 1.2 mm.

It is possible that the gel films of the present invention can contain non-thermoreversible gums. However, so as not to adversely impact the homogeneous and thermoreversible nature of the gel films of the present invention, such non-thermoreversible films should be present in an amount of less than 50% by weight of the alginate, preferably, less than 40%, more preferably, less than 30%. Examples of such nonthermoreversible gums include e.g., crosslinked and partially crosslinked gums such as calcium set (crosslinked) pectins or alginates. Calcium reactive alginates and pectins, as well as their less refined forms, are considered as thermoreversible gums in the absence of divalent cations.

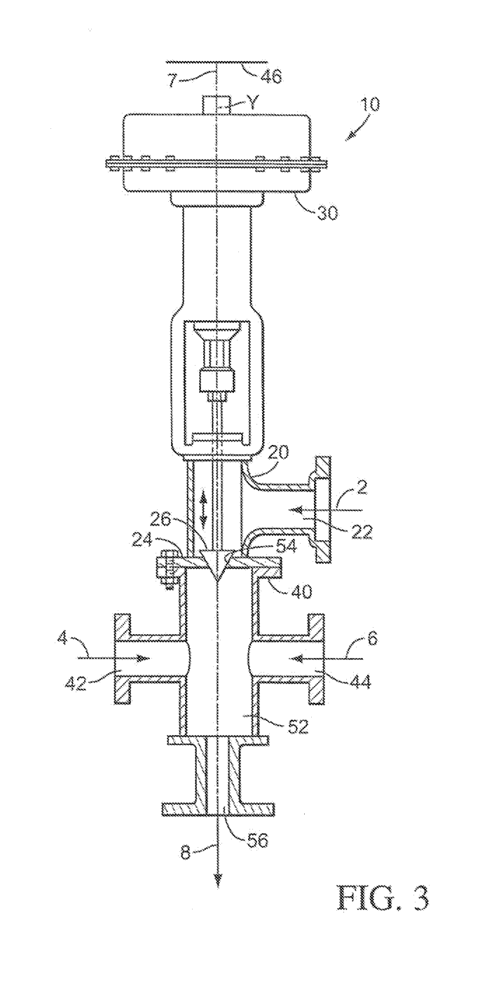

The films of the present invention are generally made from a process utilizing an apparatus that enables sufficiently high shear, temperature (above the gelling temperature) and residence time so as to provide a homogeneous molten mass of the composition and allow formation of the gel upon cooling. This is generally accomplished in the apparatus by heating, hydrating, mixing, solubilizing and optionally de-aerating the composition. Such apparatus include but are not limited to Ross mixers, Stephan processors, conventional jet cookers, extruders and the fluid mixing apparatus as set forth in FIG. 3. Ross mixers, Stephan processors, extruders and conventional jet cookers are readily available commercially. Prior to cooling, the molten mass may optionally be fed to at least one of a pump, mixer or devolatilizer; e.g., s an extruder. An extruded molten mass can also be directed to a film forming or shaping device (e.g. spreader box, as used in a capsule forming machine) that aids in the uniform casting of a continuous film, or, through a die that allows a direct formation of a film from the molten mass delivery equipment. Care must be taken to maintain the molten mass above the initiation of restricted flow/gel structure formation. Insulated and pre-heated (to maintain proper temperatures) transfer hoses may be used to insure molten mass flow until desired gel film formation is initiated on the casting rolls or at other film formation points, such as an extruder (restrictive flow, film forming device) or die. Additional processing methods (such as pre-heating the discharge/plunger-like head as seen in a Ross process system) can force (by pressure) the molten mass through the transfer hoses mentioned above. Additional insulation can help maintain molten mass temperatures through the use of a Teflon disk initially placed upon the molten mass surface immediately after removing the mixing device. In addition, the feeder hoses can be introduced to the heat controlled molten mass feeder (casting) boxes located on a capsule machine either directly to the boxes or through an optional modification of the feeder boxes which introduces a top half enclosure/cover that helps maintain molten mass temperatures within the feeder box, reduces moisture loss, and maintains uniform (center) filling of the box during the extended process of forming films for capsules. It is understood that other methods of maintaining molten mass temperatures can be used to form films for capsules. This includes, but is not limited to, extrusion of the molten mass through dies/orifices into films that: can be immediately fed into the capsule forming apparatus, stored at temperatures that maintain proper film conditions (to form capsules) until needed, or dried to desired moisture, solids and texture levels, until needed. Such dried films have the property of re-absorbing water (water is introduced by any means) throughout its gel film matrix and can be rehydrated when needed, for example, to make soft capsules or other solid forms. Moisture is, introduced to the film until a desired moisture content and strength/texture is reached that will allow the film's introduction into a capsule machine to make soft capsules.

As used herein, a “fluid mixing apparatus” refers to the apparatus in FIG. 3. FIG. 3 illustrates a fluid mixing apparatus 10. The fluid mixing apparatus 10 is arranged to mix steam 2 with a first fluid or slurry 4 and a second fluid or slurry 6 to produce a molten mass or slurry mixture 8.

The fluid mixing apparatus 10 comprises a first housing 20 having a first inlet 22 through which the steam 2 enters the housing 22, a nozzle end 24 from which the steam 2 exits the housing 20, and a nozzle valve or stem 26 disposed at the nozzle end 24. An actuator means 30 is connected to the first housing 20 for controlling the exit rate or exit pressure of the first fluid 2 at the nozzle end 24. The actuator means 30 may be of the type manufactured by Fisher Controls U.S.A.

The fluid mixing apparatus 10 further comprises a second, mixing housing 40 coupled to the first housing 20 at the nozzle end 24 of the first housing 20. The second housing 40 includes a second inlet 42 through which the first fluid 4 enters the second housing 40, and a third inlet 44 through which the second fluid 6 enters the second housing 40. The inlets 42 and 44 are disposed downstream of the first inlet 22. As shown in FIG. 3, the second inlet 42 and third inlet 44 are disposed in a common plane and spaced apart radially from each other, most preferably directly opposite (i.e., 180° apart) about the central axis Y of the mixing apparatus 10. The second housing 40 defines a generally cylindrical mixing chamber 52 that in turn defines a flow passage extending along the axial length of the mixing chamber 52 from an entry end 54 of the mixing chamber 52 to an exit end 56 of the chamber 52. The nozzle valve 26 is movable by the actuator 30 between seated and unseated positions at the entry end 54 to control the flow rate of steam 2 into the mixing chamber 52.

The nozzle end 24 of the first housing 20 directs the steam 2 into the entry end 54 of the mixing chamber 52. The second inlet 42 and the third inlet 44 radially direct the first fluid 4 and second fluid 6, respectively, into the mixing chamber 52. The steam 2, first fluid 4 and second fluid 6 are mixed in the mixing chamber 52 to form a molten mass or mixture 8 which exits the mixing chamber 52. The molten mass 8 then may be shaped into a shaped article or formed into a film, such as by casting the mixture 8 onto a cooling drum or by passing the mixture 8 through an extruder.

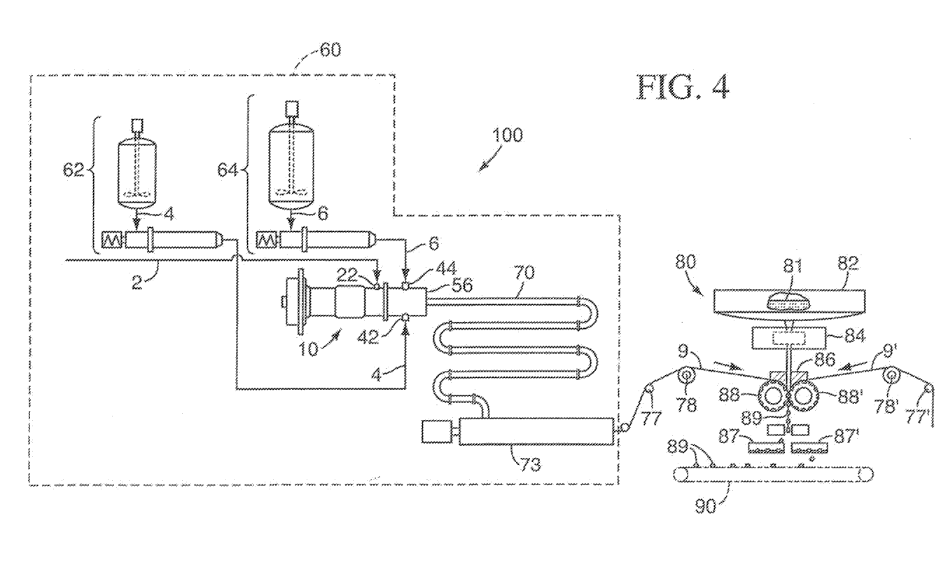

Referring next to FIG. 4, a system 100 for making films and capsules with the fluid mixing apparatus 10 includes a film preparation unit 60 for preparing and supplying a film 9, and a capsule machine 80 for forming capsules 89. The film preparation unit 60 includes: the fluid mixing apparatus 10; a first fluid supply means 62 for supplying the first fluid 4 to the fluid mixing apparatus 10; a second fluid supply means 64 for supplying the second fluid 6 to the fluid mixing apparatus 10; a slurry mixture supply path 70 for supplying the molten mass or slurry mixture 8 from the fluid mixing apparatus 8 to a shaping apparatus; an optional extruder 73 in fluid communication with the mixture supply path 70 that extrudes the mixture 8 into a film 9; a capsule machine 80 for forming capsules 89; and a conveyor belt 90 for transporting the filled capsules 90 to a subsequent process, such as drying or packaging. The extruder 73 may be of the type manufactured by Wenger or Clextrel.

The capsule machine 80 may be a conventional rotary die capsule machine of the type manufactured by R.P. Scherer Technologies of Paradise Valley, Nev. As shown in FIG. 4, the capsule machine 80 includes a capsule product storage tank 82 that holds a capsule product 81 to be encapsulated. The capsule product 81 may include liquid, semi-liquid or powder pharmaceuticals, vitamins, nutritional supplements, paint balls, pigments, agricultural actives and pre-measured food additives. The capsule machine 80 may be coupled to one or more rollers 77, 77′ and 78, 78′ so that the films 9, 9′ may be drawn into the capsule machine 80. The film 9 is fed between heater block 86 and roller die 88. Portions of the film 9 are drawn by vacuum into recesses formed in the surfaces of the rotary die 88. An amount of the capsule product 81 is delivered into the compartment formed in the film 9 by the vacuum action. Further rotary motion of the dies 88, 88′ seals the films 9, 9′ together in the nip between the rotary dies 88, 88′. Filled capsules 89 drop into bins 87, 87′ and are presented to conveyor 90 for drying and packaging.

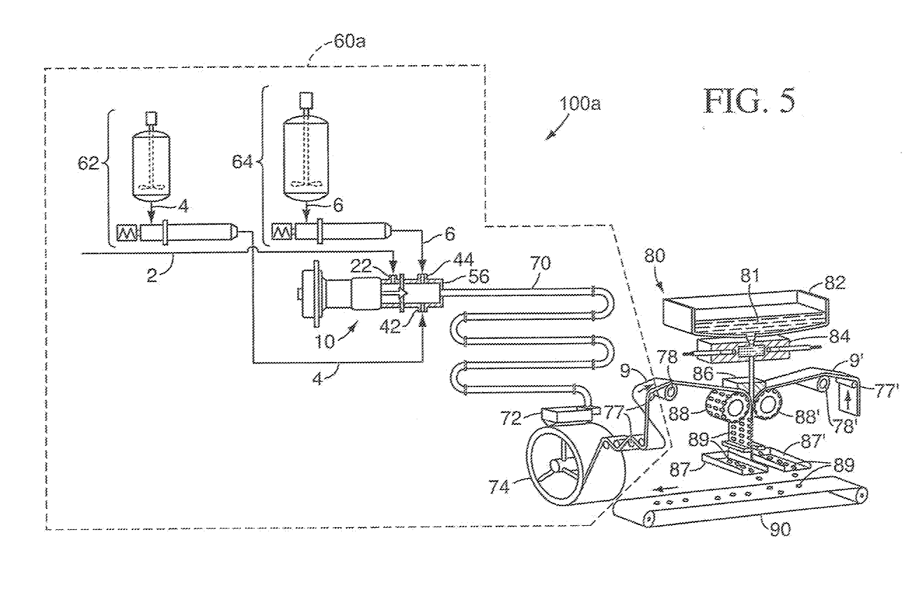

Referring next to FIG. 5, a capsule making system 100a is similar to that shown in FIG. 4, wherein like reference characters refer to like elements. In FIG. 5, however, the film preparation unit 60a includes an optional spreader box 72 and an optional cooling drum, or casting drum 74 in place of the extruder 73 of the system in FIG. 4. The system 100a includes a fluid mixing apparatus 10 and a mixture supply path 70 to direct the slurry mixture 8 away from the fluid mixing apparatus and to the spreader box 72. The spreader box 72 spreads the mixture 8 onto the casting drum 74. The film 9 is formed on the casting drum 74 as the mixture 8 cools. Thereafter, the film 9 is fed to the capsule machine 80. The film 9′ preferably is formed in the same manner as the film 9 by a second film preparation unit (not shown).

The fluid mixing apparatus 10 is adapted to produce a mixture for forming a film, more particularly an edible film for making edible capsules or strips. Incompatible film components generally are placed in different fluid inlet streams so that such incompatible components come together in the first instance at the interface of the steam injection within the mixing chamber 52 of the fluid mixing apparatus. While FIG. 3 shows inlets for steam, and first and second fluids, one or more additional inlets for one or more additional fluids may be provided. Preferably, the housings 20, 40 and other components of the fluid mixing apparatus 10 are constructed of high-grade stainless steel.

As another aspect of the invention, it is noted that the molten mass need not necessarily reach homogeneity in step (i). That is, homogeneity of the molten mass can be obtained prior to or after feeding the molten composition into at least one of the mixer, pump or devolatilizer provided the molten mass reaches homogeneity prior to gelling.

Since the gel films of the present invention have been shown to have dry film strengths of, for example, at least 2,500 grams, they are well suited to make soft capsules. Thus, the present invention is also directed to soft capsules made from the homogeneous, thermoreversible alginate gel films of the present invention, as well as methods of making such soft capsules.

The process for making soft capsules from the alginate gel films of the invention includes the use of any conventional encapsulating apparatus, e.g., a conventional rotary die apparatus or concave stamping die. For example, once the molten mass of the present invention has been made, it can be cast onto drums, cooled and then fed between rotary encapsulation dies where the films are heated again, filled, sealed and cut. For a good description of this conventional process, see WO 98/42294. Alternatively, and as a benefit of the present invention over conventional soft capsule processes, the use of the high shear apparatus disclosed above allows the molten mass to be sufficiently hydrated, applied to drums as they are cooling and then fed into conventional encapsulating apparatus for filling, sealing and cutting. This continuous type process can be used to eliminate the step of having to reheat fully gelled and cooled films for capsule preparation. The above rotary die process can be used to make soft capsules of the invention having any desired shape.

After cooling the molten composition to form the semi-gelled or fully gelled films of the invention, such films may have a moisture content of less than 50%, less than 25%, less than 15% by weight of the total components in the film prior to making soft capsules.

The fill materials for the soft capsules can be any materials widely used in the above rotary die process, including pharmaceutical ingredients, agricultural ingredients, nutraceutical ingredients, veterinary ingredients, foods, cosmetics, etc. and can be a liquid (including emulsions), suspension, dispersion, etc.

The present invention is also directed to a solid form comprising a fill material encapsulated by the homogeneous, thermoreversible gel film of the present invention. One type of such solid form is a hard capsule. Hard capsules, as used herein, refer to those solid forms that are conventionally used, e.g., in the pharmaceutical industry whereby two half shells are formed, a fill material, usually a powder, is placed in the shells and the two halves are placed together to form the hard capsule. The process for making such hard capsules would typically involve dipping metal pins or bars into the molten composition of the present invention and allowing the gel film to form around the pins. The gel films are dried and then removed from the pins. These processes are well known in the industry as methods of making hard capsules. The fill materials for the hard capsules can be any fill materials commonly used in such dosage forms. Generally, the fill materials can be liquids or solids such as powders. The fill materials can be a pharmaceutical ingredient, agricultural ingredient, nutraceutical ingredient, veterinary ingredient, food, cosmetic ingredient, etc.

The solid form may also encapsulate a powder, tablet, caplet, microcapsule or capsule in accordance with known techniques. For example, encapsulating a hard capsule with the gel film of the invention would allow for safety seal/tamper resistant capabilities.

The gel film can also be used to modify the dissolution profile of the dosage forms. For example, gel films of the invention can contain added components that can create solid dosage forms having immediate release, enteric or delayed release capabilities. Definitions of “immediate release”, “delayed release” and “enteric” can be found in the U.S. Pharmacopeia and are incorporated herein by reference.

The present invention is now described in more detail by reference to the following examples, but it should be understood that the invention is not construed as being limited thereto. Unless otherwise indicated herein, all parts, percents, ratios and the like are by weight.

### EXAMPLES

Unless otherwise indicated, the following procedures were used to prepare and evaluate the materials and films in Examples 1-3. The Stephan UMC5 processor is a laboratory scale mixing device that provided suitable high shear mixing, heating, and de-aerating of the formulations that were cast as films in the laboratory. A suitable batch size used was 1500 grams.

An aqueous starch dispersion was prepared by dissolving any salts/buffers and pH modifiers in deionized water. The starch and/or maltodextrin (M100) were added and mixed until dissolved/dispersed. Pure Cote® B760 starch is available from the Grain Processing Corporation of Muscatine, Iowa. A hydrocolloid mixture was prepared in the Stephan UMC5 processor by premixing the plasticizers until uniform, and adding the preblended dry hydrocolloids portionwise while mixing for about 30 seconds at 200 rpm after each addition. Sorbitol Special and glycerin were used as plasticizers. Sorbitol Special is an aqueous solution of sorbitol and sorbitol anhydrides at 76% solids supplied by SPI Polyols, Inc. (New Castle, Del.).

The starch dispersion was added to the non-aqueous hydrocolloid mixture and mixed at 300 rpm for 5 minutes. The mechanical agitation was increased to 2100 rpm and the mixture was heated to 85° C. to 95° C. with mixing. When the target temperature was achieved, the mixture was stirred for 30 minutes, then the sample was held under vacuum (50-60 bars) with continued agitation for an additional 45 minutes.

When the hold time under vacuum at temperature has been completed, the sample was poured into a preheated wide mouth quart Mason jar. Temperature and pH were recorded. Viscosity was measured on the hot sample using an Brookfield LVF viscometer.

A small portion of the sample was set aside and refrigerated usually overnight prior to measurement of gel/melt properties and solids using an Atago E series hand held refractometer (Gardco, Pompano Beach, Fla.). The melt temperature was determined by placing a small piece of the refrigerated gel on a wire stand placed within a test tube so that the sample does not contact the wall of the test tube. The test tube was covered with aluminum foil with a small hole to allow measurement of the gel temperature using a digital Tempermeter probe. The test tube was immersed in the heating bath so that the piece is below the surface of a hot water bath (maintained at approximately 100° C.). A silicone oil bath was used for samples that had melt temperatures above 90° C. The melt temperature was recorded when the gelled sample became wet in appearance, softened and could be stirred (a temperature range was noted). Once the sample had melted, the test tube was transferred to a second beaker containing cold tap water (15° C.). The temperature probe was used to record the temperature as the sample was cooled and to probe the sample surface to determine whether the sample had begun to gel. The gel temperature was the temperature upon cooling where the sample no longer flowed to fill in an indentation made by the probe.

The hot sample was then cast, using a draw down bar with a gap set at 3 mm clearance, onto 177 mm by 177 mm by 5 mm metal plates which were pre-sprayed with PAM (lecithin) to facilitate easy removal of film material. The gel coated plates were covered to avoid loss of moisture on the cast film and refrigerated (less than 8° C.) for at least one-half hour prior to removal of the film for testing. Refrigeration is not required for film formation. Dried film strips were prepared by drying the coated plates in a 40° C. forced air/fan oven. Films dried 2 hours at 40° C. gave an intermediate solids of about 60% while films dried overnight at 40° C. typically gave solids of 80% or higher. Test properties were measured at room temperature (about 20° C.) unless otherwise indicated. The percent of solids in the dried film was determined by difference in weight between the cast film using its formulated solids and the dried film. Break force strengths were measured on the cast and dried film strips, using a Texture Analyzer TA-108S Mini Film Test Rig.

Unless otherwise indicated, Maltrin M100 was obtained from Grain Processing Corporation, Pure-Cote B760 was obtained from Grain Processing Corporation, Sorbitol Special was obtained from SPI Polyols and Glycerin was obtained from VWR (EP/USP grade).

### Example 1

Table I lists compositions and properties of gel films prepared using blends of sodium alginates with low viscosity guar Edicol ULV 50 obtained commercially from Indian Gum Industries Ltd. Protanal® LFR 5/60, Protanal® LF 20/40 and Protanal® SF 120 RB are sodium alginates available from FMC Corporation (Philadelphia, Pa.).

TABLE I

| | | |
|---|---|---|
| Ex-1-1 | Ex 1-2 | Ex 1-3 |

Ingredient (in gms.)

| | | | |
|---|---|---|---|
| Water | 836.3 | 836.3 | 836.3 |
| LFR 5/60 | 40.5 | 0 | 0 |
| LF 20/40 | 0 | 40.5 | 0 |
| SF120 RB | 0 | 0 | 30 |
| Guar ULV50 | 49.5 | 49.5 | 45 |
| Starch B760 | 220.8 | 220.8 | 220.8 |
| M100 | 0 | 0 | 15.0 |
| Sorbitol SP | 264.4 | 264.4 | 264.4 |
| Glycerin | 88.2 | 88.2 | 88.2 |
| Total weight | 1500.0 | 1500.0 | 1500.0 |
| Temp, ° C.* | 90 | 94 | 93 |
| Viscosity, mPas* | 31,650 | >50,000 | >50,000 |
| Gel, ° C. | 50 | RT | RT |
| Melt, ° C. | 70-71 | 95 | 87 |
| PH | 5.6 | 5.5 | 5.6 |

Cast Film

| | | | |
|---|---|---|---|
| Solids, est | 40% | 40% | 40% |
| BF (g) | <40 | 102 | 110 |

Dried film (2 hrs@ 40° C.)

| | | | |
|---|---|---|---|
| Solids, est | 60% | 64% | 67% |
| BF (g) | 617 | 1250 | 1126 |

Dried film (16 hours, 40 C.)

| | | | |
|---|---|---|---|
| Solids, est | 80% | 80% | 94% |
| Avg film thickness (mm) | 0.53 | 0.89 | 0.51 |
| BF (g) | 4780 | 7701 | 10,850 |

*Temperature and viscosity of molten mass prior to casting

All the above formulations showed sufficient dry film strength for use in soft capsule manufacture, though some showed greater strengths than others. Increasing the molecular weight of the sodium alginate contributed to the overall film structure and resulted in increased film strength (Examples 1-1, 1-2 and 1-3). The melt temperatures above were measured by heating the gel films using an oil bath. When heated, they soften and could be stirred. When allowed to cool, the gel formed above room temperature.

### Example 2

Two potassium alginates were used: KAHG was the potassium salt of alginic acid extracted from Laminaria hyperborea and contains a high level guluronic acid (G) units. KAHM was a potassium salt of alginic acid extracted from Lessonia nigrescens and has a high level of mannuronic (M) units. The viscosity of the KAHG and KAHM were 5 cps and 12 cps, respectively, when measured in a 1% solution of water at 25° C.

Potassium cation associated with the alginate is beneficial for the initiation of the structuring of kappa carrageenan and/or kappa-2 carrageenan with the alginate into a homogeneous cast & set film. The kappa carrageenan was an alkali processed, clarified extract of Kappaphycus alayerei (Eucheuma cottonii). All hydrocolloids used had low levels of divalent cations as shown in Table II.

TABLE II
Cation Content of Hydrocolloids

| | Test | | |
|---|---|---|---|
| Viscosity | KAHG | KAHM | Kappa Cgn |
| Mg, % | 0.07 | 0.12 | 0.09 |
| Ca, % | 0 | 0 | 0.06 |
| K, % | 15.73 | 16.01 | 2.60 |
| Na, % | 0.63 | 0.59 | 5.45 |

Table III presents compositions and properties of films formed using mixtures of potassium alginates and other film formers such as kappa carrageenan and low viscosity guar gum available as Edicol ULV 50 from Indian Gum Industries.

TABLE III
Films Using Low Viscosity Guar, Potassium Alginates and Carrageenan

| | | | | | |
|---|---|---|---|---|---|
| Ex 2-1 | Ex 2-2 | Ex 2-3 | Ex 2-4 | Ex 2-5 | Ex 2-6 |

Ingredient (g)

| | | | | | | |
|---|---|---|---|---|---|---|
| Water | 836.3 | 836.3 | 836.3 | 836.3 | 836.3 | 836.3 |
| KAHG | 60 | 60 | 40.5 | 10.5 | 0 | 0 |
| KAHM | 0 | 0 | 0 | 0 | 60.0 | 10.5 |
| Kappa Cgn | 30 | 30 | 0 | 30 | 30.0 | 30 |
| Guar | 0 | 0 | 49.5 | 49.5 | 0 | 49.5 |
| Starch B760 | 220.8 | 220.8 | 220.8 | 220.8 | 220.8 | 220.8 |
| Sorbitol SP | 264.4 | 264.4 | 264.4 | 264.4 | 264.4 | 264.4 |
| Glycerin | 88.2 | 88.2 | 88.2 | 88.2 | 88.2 | 88.2 |
| Total weight | 1500.0 | 1500.0 | 1500.0 | 1500.0 | 1500.0 | 1500.0 |
| Temp, ° C.* | 90 | 90 | 88 | 90 | 93 | 92 |
| Viscosity, mPas* | 26,500 | 28,650 | 24,800 | 28,250 | 42,650 | 31,250 |
| Gel, ° C. | 42 | 41 | 50-51 | 53 | 39 | 55 |
| Melt, ° C. | 60-65 | 62-67 | 60-61 | 70-74 | 60-63 | 65-69 |
| pH | 7.6 | 7.2 | 6.3 | 5.6 | 7.4 | 5.9 |

Cast Film

| | | | | | | |
|---|---|---|---|---|---|---|
| Solids, est | 40% | 40% | 40% | 40% | 38% | 41% |
| BF (g) | <40 | <40 | <40 | 188 | <40 | 185 |

Dried film (2 hrs@ 40° C.)

| | | | | | | |
|---|---|---|---|---|---|---|
| Solids, est | 66% | 62% | 63% | 64% | 62% | 66% |
| BF (g) | 370 | 248 | 445 | 1811 | 502 | 1265 |

Dried film (16 hours, 40 C.)

| | | | | | | |
|---|---|---|---|---|---|---|
| Solids, est | 81% | 79% | 85% | 80% | 77% | 80% |
| Avg film | 0.83 | 0.76 | 0.56 | 0.60 | 0.56 | 0.59 |
| thickness | | | | | | |
| (mm) | | | | | | |
| BF (g) | 3826 | 4253 | 4144 | 7960 | 6918 | 8301 |

*Temperature and viscosity of the molten mass prior to casting

All the above formulations showed sufficient dry film strength for use in soft capsule manufacture, though some showed greater strengths than others. Results indicate beneficial interaction by combining potassium alginate, either high guluronic or high mannuronic, or combinations thereof, with kappa carrageenan and guar. Further variation of weight ratios of alginate and second film formers and process variations can also be used to, again, either cast as a high solids (>80%) molten mass, formed, and cooled gel film as is or for further processing.

### Example 3

Table IV lists compositions and properties of gel films prepared using propylene glycol alginate and kappa carrageenan. Protanal® ester BV4830 is a propylene glycol alginates available from FMC Corporation (Philadelphia, Pa.). HEC is hydroxyethylcellulose. Kappa carrageenan is described above in Example 2. The viscosity of the kappa carrageenan is approximately 10 to 15 mPa-s when measured in a 1.5% kappa solids 0.10 molar sodium chloride solution at 75° C.

TABLE IV
Propylene Glycol Alginate-based Blend Compositions

| | | |
|---|---|---|
| 3-1 | 3-2 | 3-3 |

Ingredient (gms.)

| | | | |
|---|---|---|---|
| Water | 840.3 | 836.3 | 840.3 |
| BV4830 | 91.2 | 49.5 | 66.0 |
| HEC | 1.9 | | |
| Kappa cgn | 24.0 | 40.5 | 54.0 |
| K citrate | 2.9 | | |
| Starch B760 | 207.8 | 220.8 | 207.8 |
| Sorbitol SP | 264.4 | 264.4 | 248.8 |
| Glycerin | 88.2 | 88.2 | 83 |
| Temp, ° C.* | 91 | 87 | 89 |
| Viscosity, mPas* | 24,800 | 6550 | 12500 |
| PH | 4.2 | 3.8 | 3.9 |
| Gel, ° C. | 59 | 42-43 | 43-44 |
| Melt, ° C. | 72-75 | 54-63 | 62-64 |

Cast film

| | | | |
|---|---|---|---|
| Solids, est. | 45% | 38% | 36% |
| BF (g) | 136 | 89 | 113 |

Dried @ 16 hours, 40° C.

| | | | |
|---|---|---|---|
| Solids | 87% | 79.8 | 86.6 |
| Avg film thickness | 0.72 | 0.68 | 0.79 |
| (mm) | | | |
| BF (g) | 8838 | 5244 | 7638 |

*Temperature and viscosity of molten mass prior to casting

All the above formulations showed sufficient dry film strength for use in soft capsule manufacture, though some showed greater strengths than others.

Table V reports compositions and film properties for kappa-2 carrageenan used in combination with propylene glycol alginate and potassium alginate. The kappa-2 carrageenan was an alkali processed, clarified extract of a mixture of Gigartina skottsbergii and Sarcothalia crispata, primarily haploid (gametophyte) plants. About 10-20% (total) of lambda- and theta-carrageenans from diploid (tetrasporophyte) plants were also present. The kappa-2 extract can be fully, partially, or not clarified when used. The viscosity of the kappa-2-carrageenan was 36 mPa-s when measured in a 1.5% kappa-2 solids 0.10 molar sodium chloride solution at 75° C.

TABLE V
Alginate Films With Kappa-2 Carrageenan

| | |
|---|---|
| Ex 3-4 | Ex 3-5 |

Ingredient (gms.)

| | | |
|---|---|---|
| Water | 834.7 | 834.7 |
| Kappa-2 Cgn | 40.5 | 54.0 |
| KAHG | 31.5 | 36 |
| BV4830 | 18.0 | 36.0 |
| M-100 | 227.3 | 227.3 |
| Sorbitol SP | 272.2 | 272.2 |
| Glycerin | 90.8 | 90.8 |
| Temp, ° C.* | 87 | 84 |
| Viscosity, mPas* | 4250 | 1050 |
| Solids | 40 | 37 |
| Melt, ° C. | 77-78 | 74-79 |
| Gel, ° C. | 54 | 52 |
| pH | 4.8 | 5.5 |

Cast film (est 40% solids)

| | | |
|---|---|---|
| BF (g) | 142 | 168 |

Dried film (est 80% solids) 16 hours, 40 C.

| | | |
|---|---|---|
| Avg film thickness (mm) | 0.67 | 0.48 |
| BF (g) | 3409 | 4004 |

*Temperature and viscosity of molten mass prior to casting

All the above formulations showed sufficient dry film strength for use in soft capsule manufacture, though some showed greater strengths than others. In example 3-4, potassium cations are supplied by the potassium alginate. The potassium cations promote carrageenan double helix formation at a temperature that allows the carrageenan to form its gel film structure. In example 3-5, the additional strength and lower processing viscosity is due to the higher level of propylene glycol alginate.

Table VI presents compositions and gel films formed from low viscosity guar Edicol ULV 50 in combination with propylene glycol alginate and other hydrocolloids. Protanal® ester BV4830 and Protanal® ester SLF3 are propylene glycol alginates available from FMC Corporation (Philadelphia, Pa.) and Kibun, respectively. The SLF-3 has a lower molecular weight compared to the BV-4830. HEC is hydroxyethylcellulose.

TABLE VI
Films using Propylene Glycol Alginate with Guar

| | | | |
|---|---|---|---|
| Ex 3-6 | Ex 3-7 | Ex 3-8 | Ex 3-9 |

Ingredient (g)

| | | | | |
|---|---|---|---|---|
| Water | 840.3 | 840.3 | 840.3 | 836.3 |
| BV4830 | 0 | 0 | 91.2 | 12.0 |
| SLF-3 | 114 | 85.5 | 0 | 0 |
| HEC | 2.4 | 1.8 | 1.9 | 0 |
| Kappa cgn | 0 | 0 | 24 | 40.5 |
| GUAR ULV 50 | 0 | 30 | 0 | 37.5 |
| Starch B760 | 207.8 | 207.8 | 207.8 | 220.8 |
| M-100 | 0 | 0 | 0 | 0 |
| Na citrate | 3.6 | 2.7 | 0 | 0 |
| K citrate | 0 | 0 | 2.9 | 0 |
| KCl | 0 | 0 | 2.4 | 0 |
| Sorbitol SP | 248.8 | 248.8 | 248.8 | 264.4 |
| Glycerin | 83 | 83.0 | 83.0 | 88.2 |
| Temp, ° C.* | 91 | 87 | 90 | 90 |
| Viscosity, mPas* | 3250 | 16,500 | 25,000 | 23,100 |
| Gel, ° C. | 34-35 | 34-38 | 43-46 | 46 |
| Melt, ° C. | 58-60 | 62-64 | 56-62 | 60-68 |
| PH | 4.4 | 4.5 | 4.3 | 4.6 |

Cast Film

| | | | | |
|---|---|---|---|---|
| Solids, est | 39% | 41 | 45% | 41.5% |
| BF (g) | <40 | <40 | 231 | 147 |

Dried film (2 hrs@ 40° C.)

| | | | | |
|---|---|---|---|---|
| Solids, est | 74% | 65% | 55% | 60% |
| BF (g) | 1877 | 355 | 842 | 592 |

Dried film (16 hours, 40 C.)

| | | | | |
|---|---|---|---|---|
| Solids, est | 85% | 77% | 78% | 80% |
| Avg film thickness (mm) | 0.67 | 0.60 | 0.75 | 0.62 |
| BF (g) | 4677 | 3317 | 9599 | 7214 |

*Temperature and viscosity of molten mass prior to casting

All the above formulations showed sufficient dry film strength for use in soft capsule manufacture, though some showed greater strengths than others.

### Example 4

The following examples show films made using the fluid mixing apparatus of FIG. 3. In these examples, Part A and Part B were pumped from separate holding tanks at ambient temperature, as two separate streams 4, 6, into two different inlet ports 42, 44 which fed the steam injection fluid mixing apparatus device 10. The two individual streams 4, 6 were combined at the interface of the steam in the mixing zone 52 of the fluid mixing apparatus 10. The separate solutions of Part A and Part B were readily pumped into the fluid mixing apparatus 10 and mixed with steam 2. The steam 2 was introduced to the mixing zone at a pressure of 120 psi. The resulting molten mass or slurry mixture 8 flowed out of the exit port 56 of the fluid mixing apparatus 10. The mixture 8 was poured onto a smooth surface and drawn down to form a homogeneous film 9.

To measure the viscosity of the mixture 8, approximately 500 ml sample of the mixture 8 was collected from the outlet 56 and poured into a jar. The temperature, pH and viscosity were measured for this sample at 95° C. A Brookfield LVF viscometer was used to measure the viscosity. An appropriate speed and spindle combination were used such that a reading could be taken. The dial reading was converted to dynamic viscosity (cP).

To measure the film strength and solids level, the molten mass 8 was collected from the outlet 56 then cast using a draw down bar, with a gap set at 3 mm, onto a stainless steel metal plate. The initial films 9 or “fresh films” were collected. Portions of the fresh films 9 were dried by placing them in a 40° C. forced air oven. Break force was measured on the fresh and dried film strips using a Texture Analyzer TA-108S Mini Film Test Rig. The percent solids were determined by measuring the difference between the initial weight of the fresh film and the final weight of the dried films.

To measure the gel temperature, a portion of the molten mass 8 was collected from the outlet 56 of the mixing apparatus 10 and transferred to a test tube. Half of the test tube remained empty. A glass thermometer was inserted into the molten mass 8. The material 8 was allowed to cool under room temperature conditions. After each degree of cooling, the thermometer was removed from the material 8. When a small, temporary indentation was observed in the surface of the mass 8, this temperature was recorded. The thermometer was re-inserted into the mass 8, which was allowed to cool further. The thermometer was removed and re-inserted at every degree of cooling until such a time as a permanent indentation formed in the mass 8, such that the indentation did not refill. The temperature at which the permanent indentation formed was recorded. The gel temperature reported was the range between the two recorded temperatures.

TABLE VII
Mixtures Containing PGA

| Example No. | 4-1 | 4-2 | 4-3 | 4-4 | 4-5 |
|---|---|---|---|---|---|
| Part A (%) | | | | | |
| Ingredients (gms.) | | | | | |
| Carrageenan B | 2.7 | 3.2 | 3.2 | 4.0 | 0.0 |
| Kappa-2 | | | | | |
| Carrageenan A | 0.0 | 0.0 | 0.0 | 0.0 | 4.0 |
| Kappa | | | | | |
| PGA | 3.3 | 3.9 | 3.9 | 4.9 | 4.9 |
| Glycerin | 22.4 | 26.5 | 26.5 | 33.5 | 33.5 |
| Part B (%) | | | | | |
| KOH | 0.0 | 0.0 | 0.1 | 0.0 | 0.0 |
| K₂CO₃ | 0.0 | 0.0 | 0.0 | 0.3 | 0.3 |
| Starch | 13.9 | 16.4 | 16.4 | 20.7 | 20.7 |
| Water | 57.8 | 50.0 | 49.9 | 36.6 | 36.6 |
| Mixing chamber | 108 | 107 | 108 | 107 | 108 |
| temp. (° C.) | | | | | |
| Outlet temp (° C.) | 102 | 102 | 102 | 101 | 102 |
| Viscosity cP | 5500 | 4650 | 2200 | 12400 | 9400 |
| (@95° C.) | | | | | |
| PH | 4.1 | 4.2 | 8.7 | 6.3 | 6.8 |
| % solids | 42 | 50 | 50 | 65 | 65 |
| Gel temp. (° C.) | 35-40 | not tested | not tested | 58-66 | 63-71 |
| wet film strength | 60 | 117 | 3874 | 337 | 822 |
| (grams) | | | | | |
| dry film strength | 2408 | 3069 | 4335 | 4561 | 4795 |
| (grams) | | | | | |

All the above formulations showed sufficient dry film strength for use in soft capsule manufacture, though some showed greater strengths than others. Table VII shows that the film former can be a combination of hydrocolloids, such as carrageenan and PGA. Additionally, salts can be added to influence film properties such as strength, gel temperature and pH.

The following Tables VIII and IX provide further descriptions of the components specified in this example.

TABLE VIII
Component Descriptions

| Name | Trade Name | Supplier | Description |
|---|---|---|---|
| Propylene glycol | Protanal BV 4830 | FMC BioPolymer | |
| alginate (PGA) | | | |
| Glycerin | | Callahan Chemical | 99.70% |
| Starch | Pure-Cote B790 | Grain Processing | |
| | | Corporation | |

TABLE IX
Carrageenan Descriptions

| | Carrageenan | | |
|---|---|---|---|
| Reference | Type | Description | Supplier |
| Carra- | Kappa | An alkali processed, clarified | FMC |
| geenan A | | kappa-carrageenan extract of | Corporation |
| | | Eucheuma cottonii with low | |
| | | divalency | |
| Carra- | Kappa-2 | An alkali processed, clarified, | FMC |
| geenan B | | low divalency extract of a | Corporation |
| | | mixture of Gigartina | |
| | | skottsbergii and Sarcothalia | |
| | | crispata, primarily haploid | |
| | | (gametophyte) plants, such | |
| | | extract being commonly known | |
| | | as “kappa-2 carrageenan”. | |
| | | Includes about 10-20% (total) of | |
| | | lambda and theta-carrageenans | |
| | | from diploid (tetrasporophyte) | |
| | | plants. Defined as the natural | |
| | | random block copolymer of | |
| | | kappa- and iota-carrageenan in | |
| | | the ratio of about 1:0 to 3.0:1 | |
| | | respectively, and has | |
| | | significantly different | |
| | | functionalities compared to | |
| | | mixing individual kappa and | |
| | | iota-carrageenan natural | |
| | | polymers at the same ratio. | |

As described and demonstrated above, the films made in accordance with this invention can be used in conventional capsule making equipment previously used for making gelatin capsules. The hydrocolloid films produced by the present invention produce less waste and provide for easier processing than gelatin-based films.

While the invention has been described in detail and with reference to specific embodiments thereof, it will be apparent to one skilled in the art that various changes and modifications can be made therein without departing from the spirit and scope thereof.

What is claimed is:

1. A homogeneous, thermoreversible gel film comprising a film forming amount of a water soluble, thermoreversible alginate and optionally at least one of a plasticizer, a second film former, bulking agent, and a pH controlling agent.

2. The gel film of claim 1, wherein said second film former is at least one of a starch, starch derivative, starch hydrozylate, cellulose gum, kappa carrageenan; iota carrageenan, kappa-2 carrageenan, polymannan gums, pullulan, gellan, pectin, an alkyl cellulose ether or a modified alkyl cellulose ether and said plasticizer is at least one polyol.

3. The gel film of claim 1, wherein said alginate is sodium alginate, potassium alginate or ammonium alginate.

4. The gel film of claim 1, wherein said alginate is at least one of propylene glycol alginate or salts of said alginate and combinations thereof.

5. The gel film of claim 1, wherein said second film former is at least one of kappa carrageenan, kappa-2 carrageenan, iota carrageenan, polymannan gums, pectin and less than fully modified versions thereof and combinations thereof.

6. The film of claim 1, wherein said alginate is present in an amount of at least 10% of the total amount of film formers in the gel film.

7. The film of claim 1, wherein said alginate is sodium alginate, potassium alginate or propylene glycol alginate, and said film further comprises at least one of hydroxyethyl cellulose, starch, starch derivative, starch hydrozylate, sodium citrate, sorbitol, glycerin or water.

8. The film of claim 1 having a break force of at least 2,500 grams.

9. The film of claim 1 having a break force of at least 4,000 grams.

10. The film of claim 1, having a break force of at least 5,000 grams.

11. The film of claim 1, having a break force of at least 6,000 grams.

12. The film of claim 1 having a solids content of at least 50% by weight of the gel film.

13. The film of claim 1 having a solids content of at least 60% by weight of the gel film.

14. The film of claim 1 having a solids content of at least 70% by weight of the gel film.

15. The film of claim 1 having a solids content of at least 80% by weight of the gel film.

16. The film of claim 1 having a solids content of at least 90% by weight of the gel film.

17. The film of claim 1, wherein said plasticizer is at least one member selected from the group consisting of glycerin, sorbitol, maltitol, lactitol, solubilized oil and polyalkylene glycols; said second film former is at least one member selected from the group consisting of a starch, starch derivative, starch hydrozylate, cellulose gum, kappa carrageenan; iota carrageenan, kappa-2 carrageenan, polymannan gums, pectin, dextrans, pullulan, gellans, an alkylcellulose ether and a modified alkyl cellulose ether; said bulking agent is at least one member selected from the group consisting of microcrystalline cellulose, microcrystalline starch, starch, starch derivatives, inulin, and starch hydrozylates; and said polyvalent cation is at least one member selected from the group consisting of calcium, magnesium, aluminum and chromium.

18. The film of claim 1, wherein said alginate is the only film former in the gel film.

19. The film of claim 1, wherein said gel film contains a second film former selected from at least one of the group consisting of kappa carrageenan, kappa-2 carrageenan and iota carrageenan.

20. Soft capsules comprising capsule walls and an encapsulated substance, wherein said capsule walls comprise the films in any of claims 1-19.

21. The soft capsules of claim 20, wherein the capsule shell has a solids content of at least 50%.

22. The soft capsules of claim 20, wherein said encapsulated substance is at least one member selected from the group consisting of pharmaceuticals, vitamins, nutritional supplements, paintballs, pigments, agriculturals, cosmetics, flavorant or food.

23. A process for making the gel films in any of claims 1-19, comprising the steps of:
(i) heating, hydrating, mixing, solubilizing, and optionally de-aerating a composition of said alginate and optionally at least one of said plasticizer, said second film former, said bulking agent and said pH controlling agent in an apparatus providing sufficient shear, temperature and residence time to form a homogeneous, molten composition, wherein said temperature is at or above the solubilizing temperature of the molten composition; and
(ii) cooling said molten composition to or below the gelling temperature of the molten composition to form the gel film.

24. The process of claim 23, wherein said molten composition is fed directly into at least one of a mixer, pump or devolatilizer.

25. The process of claim 23, wherein said apparatus is a Ross mixer, extruder, Stephan processor, jet cooker or fluid mixing apparatus.

26. A process for making soft capsules of claim 20 containing the gel films in any of claims 1-19 comprising the steps of:
(i) heating, hydrating, mixing, solubilizing, and optionally de-aerating a composition of said alginate and optionally at least one of said plasticizer, said second film former, said bulking agent and said pH controlling agent in an apparatus providing sufficient shear, temperature and residence time to form a homogeneous, molten composition, wherein the temperature is maintained at or above the solubilizing temperature of the molten composition; and
(ii) making soft capsules directly from said molten composition or allowing said molten composition to cool to its gelling temperature or below and thereafter making soft capsules therefrom.

27. The process of claim 26, wherein said apparatus is a Ross mixer, extruder, Stephan processor, jet cooker or fluid mixing apparatus.

28. The process of claim 26 wherein said molten composition is fed directly into a mixer, pump or devolatilizer.

29. A solid form comprising a fill material encapsulated by the homogeneous, thermoreversible gel film in any of claims 1-19.

30. The solid form of claim 29, wherein said fill material is a powder, tablet, caplet, microcapsule or capsule.

31. The solid form of claim 29, wherein said solid form is a hard capsule.

32. The film of claim 1 having a polyvalent cation level of 5.0% or less based on the dry weight of the alginate in the gel film, wherein said polyvalent cation is not magnesium.

33. The film of claim 32 wherein said polyvalent cation level of 2.0% or less based on the dry weight of the alginate in the gel film and said polyvalent cation is not magnesium.

34. Soft capsules comprising capsule walls and an encapsulated substance, wherein said capsule walls comprise the films in any of claims 32-33.
